(19) United States
(12) Patent Application Publication
(10) Pub. No.: US 2014/0148417 A1
(43) Pub. Date: May 29, 2014

(54) COMPOSITIONS COMPRISING A FILLER PRODUCT AND A COMPOUND OF THE TETRACYCLINE FAMILY USED AT A SUBANTIMICROBIAL DOSE

(71) Applicant: Guy Bouvier, Biot (FR)

(72) Inventor: Guy Bouvier, Biot (FR)

(73) Assignee: GALDERMA RESEARCH & DEVELOPMENT, Biot (FR)

(21) Appl. No.: 13/880,945

(22) PCT Filed: Oct. 24, 2011
(86) PCT No.: PCT/EP2011/068517

§ 371 Date: Jul. 3, 2013

Related U.S. Application Data
(60) Provisional application No. 61/344,846, filed on Oct. 22, 2010.

(30) Foreign Application Priority Data
Oct. 22, 2010 (FR) 1058649

(51) Int. Cl. A61K 8/42; A61Q 19/00; A61Q 19/08

(52) U.S. Cl. CPC A61K 8/42; A61Q 19/08; A61Q 19/00
USPC 514/152

(57) ABSTRACT

A combination product is described. The combination product can include composition “A”, which can have at least one filler product and a composition “B”, which can have at least one compound of the tetracycline family used at a subantimicrobial dose for the treatment of skin aging and scars. Compositions “A” and “B” can be used simultaneously, separately or sequentially over time. Also described, is a single pharmaceutical composition having, in the same physiologically acceptable medium, at least one filler product and at least one compound of the tetracycline family used at a subantimicrobial dose.

The present invention relates to compositions for topical and/or parenteral application comprising, in a physiologically acceptable medium, at least one filler product and at least one compound of the tetracycline family used at a subantimicrobial dose. The invention also relates to the use of these compositions especially for treating skin aging, in particular for treating by filling wrinkles, fine lines, fibroblast depletions, dehydration of the skin and any scars.

Skin aging is one of the most visible modifications of the senescence process. In addition, the skin is exposed to many factors capable of accelerating this physiological process.

A distinction is made between two different types of skin aging. Firstly, intrinsic (or physiological) aging, which is evaluated more easily on areas of skin that are not normally exposed to the sun, and, secondly, extrinsic aging caused by the interaction of environmental factors, in particular UV rays. These environmental factors have a much more marked effect on the parts of the body exposed to the sun, especially in individuals with a fair phototype. The term actinic aging is then also used. Other factors, such as dietary habits, smoking, excessive alcohol consumption, chronic diseases and endocrine gland dysfunctions, also contribute to this aging.

During intrinsic skin aging, the horny layer is relatively unmodified. The epidermis is atrophic and the dermal-epidermal junction is flattened, such that the adhesion to the dermis is weaker, facilitating the formation of bubbles. The thickness of the dermis is very much reduced; there are fewer blood vessels. Fewer fibroblasts are also observed and their biosynthetic and proliferative capacities are reduced. The elastic fibres first undergo modifications, and subsequently disappear.

As regards extrinsic aging, an irregular, sometimes atrophic, sometimes hyperplastic, epidermis is observed, with signs of disorganization and of dysplasia. There are more melanocytes in certain areas, and fewer in others. The distribution of melanin in the epidermis is also irregular, subsequent to melanosome transfer problems. The number of Langerhans cells decreases. The small blood vessels are first dilated, and then become thinner and atrophy.

Wrinkles are the most visible signs of aging. A distinction can be made between several types, in particular superficial and deep wrinkles. Deep wrinkles appear to be due to dermo-hypodermal modifications, whereas superficial wrinkles could be explained by dermal and possibly epidermal modifications. Wrinkles are especially due to the loss of elasticity of the skin. The collagen present in the dermis fragments, and this fragmentation leads to a loss of structural integrity and also fibroblast dysfunction (Fisher et al, Looking older, Arch Dermatol. 2008; 144(5): 666-672). The damage to the subepidermal elastic network gives rise to superficial laxity of the aged skin and to folding of its surface. The destruction of the elastic fibres in the reticular dermis is responsible for the loss of elasticity and of the ability of the skin to return its shape after stretching. A suitable treatment will be possible according to the type, the intensity and the topography of the wrinkles.

The skin contains 20% of all the water contained in the human body and 70% of the water in the skin is concentrated in the dermis. It plays a fundamental role by contributing to the mechanical properties of the dermis and to the physiological functioning of the skin, in particular to the integrity of the skin barrier. Hyaluronic acid, which is synthesized by the fibroblasts and the keratinocytes, is a major constituent of the extracellular matrix which has an important structural function, which makes it one of the key components for maintaining dermal density and therefore the firmness of the skin. It is also a real water sponge essential for maintaining hydration. The synthesis and the quality of hyaluronic acid decreases over the years, causing dehydration, sagging and loss of firmness of the skin. It is therefore essential to maintain a good level of hydration of the skin, in order to limit skin aging.

Some scars are pathological; among the latter, mention may be made of recessed scars and raised scars. Recessed scars are hypotrophic scars which can be unsightly, more particularly when they affect the face in the case of acne scars.

Over the past few years, there has been an enormous amount of progress in the treatment of unsightly skin modifications related to aging and to scars. A relatively large number of natural or synthetic substances have already been described as dermal implants, i.e. as substances injected directly into the skin, in order to remedy skin modifications resulting from aging, from trauma or from diseases.

Such therapeutic techniques are in particular the local injection of deactivated botulinum toxin (Botox®) or the use of laser techniques. These various treatments are not exclusive and combining them has even been recommended. Currently, many dermal implants are used, but none has yet been considered ideal in the context of a safe and healthy tissue augmentation (Naoum C, Dasiou-Plakida D. Dermal filler materials and botulin toxin Int J Dermatol. 2001 October; 40 (10): 609-21).

Moreover, many side effects of these implants have been observed, such as haematomas, erythema, oedema (which can occur rapidly, i.e. within a day or week following application of the product); infections and inflammations (semi-delayed side effects, occurring within 1 to 3 weeks following application of the product); or else allergies or pigmentations of the skin as a reaction to the product.

Indeed, the inflammation triggered by administration of these implants activates the immune system, in particular through activation of macrophages, resulting in the release of certain mediators and of free radicals which promote degradation of the implants.

Conventionally, doxycycline and other tetracyclines are normally used for their broad-spectrum antibiotic properties for treating numerous bacterial infections.

Given the aforementioned, one problem that the invention proposes to solve is that of producing compositions which make it possible to effectively treat skin aging while at the same time substantially reducing the side effects resulting directly or indirectly from the induction of the inflammatory reaction, and increasing the lifespan of the filler products by decreasing the degradation processes directly related to the inflammatory reaction or related to the activation of enzymes involved in the extracellular matrix degradation process (metalloprotease, protease, collagenase, elastase, in particular).

The present invention makes it possible to prevent and treat skin aging, in particular wrinkles and fine lines, with improved effectiveness and much reduced side effects.

The subject of the present invention is thus the specific combination of at least one filler product and at least one compound of the tetracycline family used at a subantimicrobial dose. Such a combination makes it possible to treat skin aging and scars, in particular by filling, in an effective and safe manner.

The first subject of the invention is therefore a combination product comprising:
a composition A comprising at least one filler product, and
a composition B comprising at least one compound of the tetracycline family used at a subantimicrobial dose,
   for the treatment of skin aging and scars, compositions A and B being used simultaneously, separately or sequentially over time.

The term “simultaneously” is intended to mean application of the filler product and of the compound of the tetracycline family used at a subantimicrobial dose at the same time.

The term “separately” is intended to mean the consecutive application of each composition, of the filler product and of the compound of the tetracycline family used at a subantimicrobial dose, over a short period of time (i.e. less than or equal to 1 h).

The term “sequentially over time” is intended to mean a consecutive application of each composition, of the filler product and of the compound of the tetracycline family used at a subantimicrobial dose, over a longer period of time (i.e. greater than 1 h and possibly ranging up to one month after injection of the filler product).

Preferably according to the invention, the compositions A and B are used simultaneously, and more preferably in one fixed combination product.

The filler product can therefore be administered to a patient independently, as is the case for composition A of the combination product. In this case, the filler product is included in a separate composition (composition A of the combination product), which can be administered simultaneously or else at a different time from that of the administration of composition B comprising the compound of the tetracycline family used at a subantimicrobial dose.

Composition A comprising the filler product could be administered topically or parenterally, for example by injection. Preferably, composition A is administered by injection.

Composition B of the combination product, comprising the chemically modified tetracycline, can be administered topically, parenterally or orally. Preferably, composition B is administered by injection or topically.

Preferably, composition A is in the form of an injectable solution and composition B is suitable for application by injection or topically. When compositions A and B are each in the form of an injectable solution, they can be administered by simultaneous coinjection, in particular using a double syringe, or else by injection of each solution one after the other, or using a combination of A and B in one injectable fixed combination product.

The combination product is preferentially used for the treatment of skin aging, and more preferentially for the treatment of wrinkles and fine lines.

The subject of the invention is also and preferably a single pharmaceutical composition, in particular for topical and/or parenteral application, comprising, in the same physiologically acceptable medium, at least one filler product and at least one compound of the tetracycline family used at a subantimicrobial dose.

The term “physiologically acceptable medium” is intended to mean a medium that is compatible with the skin, the mucous membranes and/or the skin appendages.

The filler product may also contain another active ingredient, for instance an anaesthetic such as lidocaine.

Said single pharmaceutical composition is preferably intended for the treatment of skin aging and of scars. Preferentially, the pharmaceutical composition is intended for the treatment of wrinkles and fine lines.

The term “skin aging” is intended to mean more particularly wrinkles, fine lines, fibroblast depletions and dehydration of the skin.

This single pharmaceutical composition can be administered topically or parenterally, for example by injection. Preferably, this single pharmaceutical composition is administered by injection. Preferably, this single pharmaceutical composition is administered using a syringe or any other injection device.

The subject of the present invention is also a kit comprising:
at least one filler product, and
at least one compound of the tetracycline family used at a subantimicrobial dose.

The kit according to the invention comprises a first composition A comprising at least the filler product, and a second composition B, distinct from the first, comprising the compound of the tetracycline family used at a subantimicrobial dose. The two compositions obtained can be packaged together or separately.

The subject of the invention is also and preferably the fixed combination of at least one filler product and at least one compound of the tetracycline family used at a subantimicrobial dose, for use thereof in the treatment of skin aging and scars.

The combination product or the single pharmaceutical composition according to the invention comprises at least one filler product.

The term “filler product” is intended to mean any product which, when it is applied in particular by injection to the skin, makes it possible to smooth out said skin. The consistency of said product is therefore more or less thick depending on the depth of the recess to be filled in.

Said filler product is in particular composed of classical filler compounds well-known by the man skilled in the art of aesthetic dermatology. By filler compounds according to the invention, it is understood collagen and its derivatives, hyaluronic acid, its salts and its derivatives, alginates, synthetic polymers, elastin and biological polymers, and mixtures thereof.

Preferably, the filler product comprises filler compounds chosen from collagen of human origin, collagen of porcine origin, collagen of bovine origin, crosslinked collagens, hyaluronic acid, its salts and its derivatives, lactic acid polymers, methacrylate derivatives, calcium phosphate derivatives, polyacrylamides, polyurethanes, polyalkylimide gels, polyvinyl microspheres, silicones, silica (SiO₂) polymers and biological polymers, and mixtures thereof.

Collagen is a fibrous protein, of approximately 300 kDa, which makes up the connective tissue in the animal kingdom. It may be of human or nonhuman origin, in particular of porcine or bovine origin. Collagen derivatives include, inter alia, crosslinked collagens. The collagen and derivatives thereof can optionally be mixed with an anaesthetic, such as lidocaine.

Filler products based on collagen and derivatives thereof are in particular the products:
Cosmoderm® and Cosmoplast® from Inamed/Allergan, which are purified human collagens mixed with lidocaine;
Zyderm® and Zyplast® from Inamed/Allergan, which are highly purified bovine collagens mixed with lidocaine; or else
Evolence® from ColBar, which is made up of crosslinked porcine collagen.

The filler product may also comprises filler compounds chosen from an alginate or salts thereof. Among the alginates that can be used, mention may be made of alginate salts, such as sodium alginate, or else crosslinked alginates, for instance the product Novabel® from Merz Aesthetics.

The filler product may also comprises filler compounds chosen from a synthetic polymer. The term “synthetic polymer” is intended to mean a system formed by a collection of macromolecules of the same chemical nature, resulting from the covalent linking of a large number of identical or different monomer units of synthetic origin, prepared by polymerization of monomer molecules. Preferably, said synthetic polymer is chosen from lactic acid polymers such as poly-L-lactic acid, methacrylate derivatives such as hydroxyethyl methacrylate (HEMA), ethyl methacrylate (EMA), or poly(methyl methacrylate) (PMMA), calcium phosphate derivatives (hydroxylapatite or tricalcium phosphate), polyacrylamides, polyurethanes, polyalkylimide gels, polyvinyl microspheres, silicones and silica (SiO₂) polymers, and mixtures thereof.

Filler products based on synthetic polymers are in particular the products:
Sculptra® from Dermik, which is a synthetic polymer of lactic acid;
Artefill® or Artecoll® from Artes Medical, which is a mixture of PMMA and bovine collagen and lidocaine;
Radiesse® from BioForm, which is calcium hydroxyapatite;
Beta-Altean® from Stiefel, which is tricalcium phosphate;
Bio-Alcamid® from SkinRx Distribution Inc., which is a polyalkylimide gel,
   Evolution® from Procytech SA, which is polyvinyl microspheres, or else
   Aquamid® from Ferrosan A/S/Contura, which is a polyacrylamide gel.

The filler product may also comprises filler compounds chosen from a biological polymer. The term “biological polymer” is intended to mean a system formed by a collection of macromolecules of the same chemical nature, resulting from the covalent linking of a large number of identical or different monomer units of natural origin or artificial origin (obtained by chemical modification of a natural polymer). Preferably, said biological polymer is chosen from polymers of methylcellulose, of agarose, of dextran or of chitosan.

Filler products based on biological polymers are in particular the products:
Reviderm Intra® from Rofil Medical International, which is a dextran polymer.

More preferentially, the filler product comprises filler compounds chosen from hyaluronic acid, a pharmaceutically acceptable salt thereof or a derivative, particularly a sodium or potassium salt.

Hyaluronic acid can be used in various forms: a salt, a derivative such as an ester or an amide, or in linear or crosslinked form. In particular, the molecular weight is conventionally between 500 kDa and 5000 kDa and the degree of crosslinking depends on the use and the site of application, especially in the wrinkle field.

Hyaluronic acid is a ubiquitous natural polysaccharide which exists in the same form from the simplest bacterium to humans. It is a polysaccharide composed alternately of D-glucuronic acid and N-acetylglucosamine, linked to one another by alternating beta-1,4 and beta-1,3 glycosidic linkages. According to Saari H et al. (Differential effects of reactive oxygen species on native synovial fluid and purified human umbilical cord hyaluronate, Inflammation 17 (1993): 403-415), the polymers of this recurring unit may be between 10² and 10⁴ kDa in size, in vivo, hyaluronic acid taken from the umbilical cord having a weight of 2500 kDa.

Hyaluronic acid represents a natural constituent of the dermis, where it plays an important role in the hydration and elasticity of the skin. However, it decreases in amount and in quality with age, leading to drying out of the skin, which becomes wrinkled. It is highly water-soluble and forms high-viscosity solutions in water. The tolerance of hyaluronic acid is very good and no immunogenicity has been associated with this substance.

The hyaluronic acid may be of human or nonhuman origin, for instance of avian or bacterial origin.

The term “pharmaceutically acceptable salt” is intended to mean a basic or acidic salt, such as, by way of nonlimiting examples, hydrochloride, hydrobromide, hydroiodide, nitrate, sulphate, bisulphate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulphonate, ethanesulphonate, benzenesulphonate, p-toluenesulphonate and pamoate (i.e., 1,1′-methylenebis(2-hydroxy-3-naphthoate)), salts, aluminium, calcium, lithium, magnesium, potassium, sodium, zinc, or diethanolamine salts. Certain compounds according to the invention can form pharmaceutically acceptable salts with various amino acids (Berge et al., 66 J. Pharm. Sci. 1-19 (1977)).

The hyaluronic acid may also be combined in the filler product with at least one dextran, in order to slow down its degradation in vivo.

Filler products based on hyaluronic acid and derivatives thereof are in particular the products:
EMERVEL®, a complete range of nine scientifically-advanced and clinically-proven hyaluronic acid dermal fillers, also available with the local anaesthetic lidocaine;
Restylane® and Perlane® from Q-Med/Galderma, or Juvederm® from Allergan/Corneal, or Prevelle Silk® from Genzyme, which are hyaluronic acids of Streptococcus bacteria;
Hylaform®, Hylaform Plus® or Captique® from Genzyme/Allergan, which are chemically modified hyaluronic acids of avian origin;
Elevess® from Anika, which is composed of hyaluronic acid of Streptococcus bacteria mixed with lidocaine; or else
Matridex® from TBMC Aesthetics, which is a gel composed of hyaluronic acid combined with dextran beads (DEAE Sephadex).

The combination product or the single pharmaceutical composition according to the invention also comprises at least one compound of the tetracycline family used at a subantimicrobial dose.

The tetracyclines according to the invention are natural or semisynthetic tetracycline compounds obtained from tetracycline, used at a subantimicrobial dose.

The term “subantimicrobial dose” is intended to mean a dose which is less than that which makes it possible to have an antibiotic activity.

Tetracycline is an antibiotic having the following formula:

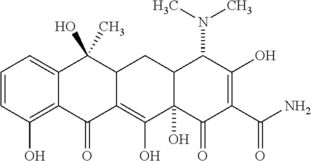 [SMILES: CN(C)[C@@H]1C(O)=C(C(N)=O)C(=O)[C@@]2(O)C(O)=C3C(=O)c4c(O)cccc4[C@@](C)(O)C3CC12]

The carbons on the tetracycline nucleus are numbered as follows:

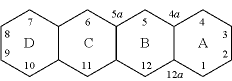

The term “compound of the tetracycline family” is intended to mean more particularly natural or semisynthetic tetracyclines, such as doxycycline, minocycline, tetracycline, tetracycline hydrochloride, oxytetracycline, chlorotetracycline, demeclocycline, etamocycline, rolitetracycline, metacycline, lymecycline, meclocycline and tigecycline.

In the present description, and unless otherwise specified, it is understood that the term “compositions according to the invention” is intended to mean compositions A and B or else the single pharmaceutical compositions, or the fixed combination composition containing both the filler product and the compound of the tetracycline family used at a subantimicrobial dose.

In the compositions according to the invention, the filler compound present in the filler product is present in proportions that can range from 0.001% to 10% by weight, relative to the total weight of the composition, preferentially from 0.1% to 5% of filler product, more preferably from 1.5% to 2.5% by weight, relative to the total weight of the composition.

In the compositions according to the invention, the chemically modified tetracycline is present in proportions that can range from 0.01% to 5% by weight, relative to the total weight of the composition, more preferably from 0.02% to 0.03% by weight, relative to the total weight of the composition.

In one preferred embodiment, the composition according to the invention is an injectable fixed combination composition. Preferably, the composition comprises a filler composed of Hyaluronic acid, in proportions that can range from 1 to 3% by weight, relative to the total weight of the composition, preferentially from 1.5% to 2.5%, more preferable from 2 to 2.5% and from 0.01% to 5% of chemically modified tetracycline, preferably from 0.02 to 0.03% by weight, relative to the total weight of the composition, in a physiological vehicle. The vehicle is preferably a physiological buffer, chosen from water, saline solution, phosphate buffer. According to the invention physiological buffer leads to a physiological pH of the composition, which can range from pH 6 to pH 8 and more preferable to pH 7.

In the preferred case of the injectable fixed combination composition, ranges of hyaluronic acid are also expressed in weight relative to total volume of the composition. Preferably, the composition comprises a filler composed of Hyaluronic acid, in proportions that can range from 15 to 30 mg per ml of the total composition, preferentially from 15 to 25 mg/ml, more preferably from 20 to 25 mg/ml and from 1 to 5 mg/ml of chemically modified tetracycline, preferably from 2 to 3 mg/ml of the total volume of the composition, in a physiological vehicle.

In the preferred case of injectable fixed combination composition, the composition is a composition having a total volume comprised between 0.8 and 5 ml, preferably 1 or 2 ml.

In the present description, and unless otherwise specified, it is understood that, when concentration ranges are given, they include the upper and lower limits of said range.

In a known manner, the compositions according to the invention may also contain the usual adjuvants well known to those skilled in the art.

The term “topically” is intended to mean external application to the skin or the mucous membranes. Topically, the compositions can be in any of the galenic forms normally used for topical administration. By way of nonlimiting example of topical compositions, mention may be made of compositions in liquid, pasty or solid form, and more particularly in the form of salves, aqueous, aqueous-alcoholic or oily solutions, dispersions of the lotion type, which optionally have two-phases, sera, aqueous, anhydrous or lipophilic gels, powders, impregnated pads, syndets, wipes, sprays, foams, sticks, compresses, washing bases, emulsions of liquid or semi-liquid consistency of the milk type, obtained by dispersion of a fatty phase in an aqueous phase (O/W) or vice versa (W/O), a microemulsion, suspensions or emulsions of soft, semi-liquid or solid consistency of the cream or ointment type, suspensions of microspheres or nanospheres or lipid or polymeric vesicles, or microcapsules, microparticles or nanoparticles or polymeric or gel patches for controlled release.

The term “parenterally” is intended to mean subcutaneous or intradermal application. By way of nonlimiting examples of parenteral compositions, mention may be made of compositions in the form of solutions or suspensions for infusion or for injection. Parenterally, the compositions may be administered using conventional syringes or double syringes (where two compositions are separated from one another by a leaktight membrane, and where the mixing takes place as the compositions leave the syringe).

In the context of a combined administration of the filler product and of the compound of the tetracycline family according to the invention, the administration frequencies may be identical or different. Advantageously, in the context of the invention, the frequency of administration of composition A injected in the form of an injectable solution can range from 4 to 24 months, preferentially from 4 to 16 months, whereas those of composition B according to the invention, administered topically, for example in the form of a cream, can range from 1 to 30 days, preferentially from 1 to 7 days.

More preferably the invention relates to the use of at least one filler product and at least one chemically modified tetracycline, in a fixed combination product, for producing a pharmaceutical composition intended for the treatment of skin aging and of scars.

The composition according to the invention is suitable for the treatment of wrinkled and/or aged skin, and is aimed in particular at preventing and/or reducing the effects thereof. The treatment of wrinkles, fine lines, fibroblast depletions, dehydration of the skin and any scars is carried out in particular by filling-in. The composition according to the invention can be applied to the skin of the face and of the body.

In particular, the composition according to the invention can be applied to the areas of the face or of the forehead that are marked by expression wrinkles or by scars, inter alia due to acne.

The invention also relates to the use of at least one filler product and at least one compound of the tetracycline family used at a subantimicrobial dose, preferably of a single pharmaceutical composition as described above or of a combination product, for producing a pharmaceutical composition intended to be used in reconstructive surgery.

The present invention will now be illustrated by means of the following examples.

### EXAMPLE 1

### Fixed Combination Composition According to the Invention

| Component | Concentration (mg/ml) | |
|---|---|---|
| Hyaluronic acid | 20 | mg/ml |
| Doxycycline | 3 | mg/ml |
| Physiological Buffer | Qs 1 | ml |

### EXAMPLE 2

### Fixed Combination Composition According to the Invention

| Component | Concentration (mg/ml) | |
|---|---|---|
| Hyaluronic acid | 20 | mg/ml |
| Lidocaine | 3 | mg/ml |
| Minocycline | 3 | mg/ml |
| Physiological Buffer | Qs 1 | ml |

### EXAMPLE 3

### Fixed Combination Composition According to the Invention

| Component | Concentration (mg/ml) | |
|---|---|---|
| Hyaluronic acid | 25 | mg/ml |
| Lidocaine | 3 | mg/ml |
| Doxycycline | 2 | mg/ml |
| Physiological Buffer | Qs 1 | ml |

### EXAMPLE 4

### Fixed Combination Composition According to the Invention

| Component | Concentration (mg/ml) | |
|---|---|---|
| Hyaluronic acid | 20 | mg/ml |
| Lidocaine | 3 | mg/ml |
| Tetracycline hydrochloride | 1.5 | mg/ml |
| Physiological Buffer | Qs 1 | ml |

### EXAMPLE 5

### Fixed Combination Composition According to the Invention

| Component | Concentration (%) |
|---|---|
| Hyaluronic acid | 2% |
| Lidocaine | 0.03% |
| Doxycycline | 0.03% |
| Physiological Buffer | Qs 100 |

### EXAMPLE 6

### Fixed Combination Composition According to the Invention

| Component | Concentration (%) |
|---|---|
| Collagen | 2.5% |
| Lidocaine | 0.03% |
| Doxycycline | 0.03% |
| Physiological Buffer | Qs 100 |

### EXAMPLE 6

### Fixed Combination Composition According to the Invention

| Component | Concentration (%) |
|---|---|
| Poly-L-lactic acid | 2.5% |
| Lidocaine | 0.03% |
| Minocycline | 0.03% |
| Physiological Buffer | Qs 100 |

1. A combination product comprising:
a composition A comprising at least one filler product, and
a composition B comprising at least one compound of the tetracycline family used at a subantimicrobial dose, for the treatment of skin aging and scars, compositions A and B being used simultaneously, separately or sequentially over time.

2. A combination product as defined by claim 1, wherein the composition is used in the treatment of skin aging.

3. A combination product as defined by claim 1, wherein composition A is in the form of an injectable solution and composition B is suitable for topical application.

4. A combination product as defined by claim 1, wherein composition A and composition B are in the form of an injectable solution.

5. A combination product as defined by claim 1, wherein composition A and composition B are in the form of a single pharmaceutical composition comprising, in the same physiologically acceptable medium, at least one filler product and at least one compound of the tetracycline family used at a subantimicrobial dose.

6. A combination product as defined by claim 4, wherein composition A and composition B are in the form of a fixed combination composition comprising, in the same physiologically acceptable medium, at least one filler product and at least one chemically modified tetracycline.

7. A combination product as defined by claim 1, wherein the filler product comprises a member selected from the group consisting of collagen, hyaluronic acid, hyaluronic acid salts, alginates, alginate salts, synthetic polymers, elastin polymers, biological polymers, and mixtures thereof.

8. A combination product as defined by claim 1, wherein the filler product is selected from the group consisting of collagen of human origin, collagen of porcine origin, collagen of bovine origin, crosslinked collagens, hyaluronic acid, alginate salts, crosslinked alginates, lactic acid polymers, methacrylate derivatives, calcium phosphate derivatives, polyacrylamides, polyurethanes, polyalkylimide gels, polyvinyl microspheres, silicones, silica (Si02) polymers, polymers of methylcellulose, polymers of agarose, polymers of dextran, polymers of chitosan, and mixtures thereof.

9. A combination product as defined by claim 1, wherein the compound of the tetracycline family is chosen from natural tetracyclines or semisynthetic tetracyclines.

10. A combination product as defined by claim 1, wherein the compound of the tetracycline family is selected from the group consisting of doxycycline, minocycline, tetracycline, tetracycline hydrochloride, oxytetracycline, chlorotetracycline, demeclocycline, etamocycline, rolitetracycline, metacycline, lymecycline, meclocycline and tigecycline.

11. A method of treating skin aging, the method comprising administering a combination product as defined by claim 1 to an individual subject in need thereof.

12. A method of treating skin aging, the method comprising administering a combination product as defined by claim 1 to an individual subject in need thereof wherein the treatment comprises treating wrinkles, fine lines, fibroblast depletions and dehydration of the skin.

13. A kit comprising:
a first composition A comprising at least the filler product, and
a second composition B, distinct from the first, comprising a compound of the tetracycline family used at a subantimicrobial dose.

14. A method of treating skin aging and scars, the method comprising administering a combination of at least one filler product and at least one compound of the tetracycline family used at a subantimicrobial dose to an individual subject in need thereof.
